(19) United States
(12) Patent Application Publication
(10) Pub. No.: US 2020/0075173 A1
(43) Pub. Date: Mar. 5, 2020

(54) Method and Apparatus for Responding to the Presence of a Communicable Condition

(71) Applicant: Hill-Rom Services, Inc., Batesville, IN (US)

(72) Inventor: John D. Christie, Batesville, IN (US)

(21) Appl. No.: 16/543,911

(22) Filed: Aug. 19, 2019

Related U.S. Application Data
(60) Provisional application No. 62/726,589, filed on Sep. 4, 2018.

(51) Int. Cl. G16H 50/80; G16H 40/20

(52) U.S. Cl. CPC G16H 50/80; G16H 40/20

(57) ABSTRACT

A method of responding to the presence of a communicable condition includes the steps of 1) acquiring a spatial/temporal trajectory of each member of a population of interest, 2) acquiring a spatial/temporal trajectory of zero or more objects of interest, 3) identifying a communicable condition associated with at least a first member of the population, 4) and, subsequent to the identifying step, assessing possible transfer of the communicable condition to one or more other members of the population. The assessment is based, at least in part, on the acquired spatial/temporal trajectories. A related apparatus includes a processor and machine readable instructions. The instructions, when executed by the processor, carry out the above enumerated steps 1, 2, 3 and 4.

### CROSS REFERENCE TO RELATED APPLICATIONS

This application claims priority to U.S. Provisional Application 62/726,589 filed on Sep. 4, 2018, and entitled “Method and Apparatus for Responding to the Presence of a Communicable Condition”, the contents of which are incorporated herein by reference.

### TECHNICAL FIELD

The subject matter described herein relates to a method and apparatus for responding to the presence of a communicable condition, such as a disease. One venue in which the method and apparatus may find use is a hospital or other health care facility.

### BACKGROUND

A hospital or other health care facility presents favorable conditions for transference of a communicable condition such as a disease (or the cause of the condition such as a virus) from one person to another.

In some cases it may be recognized before a patient enters the facility that he suffers from a communicable condition. In such cases precautions can be taken to prevent further transmission of the condition. Such precautions may include isolating the patient from other patients and ensuring that individuals who must interact with the patient, such as nurses and meal service staff, are forewarned of the patient's condition and take protective measures to avoid acquiring the condition or serving as a carrier thereof.

However measures such as those described above may not be completely effective. For example a staff member may forget to wear a face mask or may have an undetected tear in a protective glove. In such cases the patient's condition may be initially transferred directly to the unprotected person, or indirectly to the unprotected person if the patient interacts with an object and the unprotected person later interacts with that same object. The unprotected person may then transfer the condition to other persons and objects.

A considerable amount of time may elapse between the time of the initial transference of the condition to the unprotected person and the time that that person is recognized as having acquired the condition. Therefore it is helpful to be able to develop an understanding, which is retrospective relative to the time of recognition, of which other persons and objects the condition may have been transferred to. Actions can then be taken to control further spread of the condition, and to assist persons who might have acquired the condition, even though those persons may still be asymptomatic.

Additional time may elapse between the time the unprotected person is recognized as having acquired the condition, and the time when a response to arrest further spread of the condition gets underway. Therefore, it is also helpful to develop a prospective understanding of any other persons and objects that the condition may have been transferred to in the time frame between recognition and response.

A similar situation arises when a patient has acquired a communicable condition, but is not recognized as having acquired the condition. At a later time, when it becomes known that the patient is affected by the condition, it is helpful to be able to develop the prospective and retrospective understandings described above.

Occurrences other than those described above (ineffectiveness of protective measures, and delayed recognition that a patient has a communicable condition) may also give rise to the need to develop an understanding of transference of a communicable condition.

Therefore, it is useful to develop methods and systems for tracking movements and interactions of people and objects in connection with controlling the transference of a communicable condition among members of a population.

### SUMMARY

A method of responding to the presence of a communicable condition includes 1) acquiring a spatial/temporal trajectory of each member of a population of interest, 2) acquiring a spatial/temporal trajectory of zero or more objects of interest, 3) identifying a communicable condition associated with at least a first member of the population, 4) and, subsequent to the identifying step, assessing possible transfer of the communicable condition to one or more other members of the population, the assessment being based, at least in part, on the acquired spatial/temporal trajectories.

### BRIEF DESCRIPTION OF THE DRAWINGS

The foregoing and other features of the various embodiments of the occupant support structure and method described herein will become more apparent from the following detailed description and the accompanying drawings in which:

FIG. 1 is schematic plan view of a hospital wing representing a domain of interest.

FIG. 2 is a space/time diagram showing a sample of the locations and movements of objects and nurses in the domain of FIG. 1 during a period of time.

FIG. 3 is a diagram showing chain transference of a communicable condition from a source or first person to a recipient or second person by way of three mediators.

FIG. 4 is a diagram showing chain transference of a communicable condition from a source or first person to a recipient or second person by way of one mediator.

FIG. 5 is a diagram showing transference of a communicable condition directly from a source or first person to a recipient or second person or, alternatively, transference of the communicable condition by way of zero mediators.

FIG. 6 is a block diagram showing a method of responding to the presence of a communicable condition.

FIG. 7 is a diagram illustrating different possible sequencings of actions of FIG. 6.

FIG. 8, is a diagram illustrating how the method of FIG. 6 associates a communicable condition with a first member of the population and with a second member of the population.

FIG. 9 is a diagram similar to FIG. 8 illustrating how the method of FIG. 6 associates a first communicable condition with a first member of the population and associates a second communicable condition with a second member of the population.

FIG. 10 is a diagram similar to FIG. 8 illustrating how the method of FIG. 6 associates a first condition and a second condition with the same member of the population.

FIG. 11 is a block diagram similar to FIG. 6 enumerating specific possible responses to the presence of a communicable condition.

### DETAILED DESCRIPTION

The present invention may comprise one or more of the features recited in the appended claims and/or one or more of the following features or combinations thereof.

In this specification and drawings, features similar to or the same as features already described may be identified by reference characters or numerals which are the same as or similar to those previously used. Similar elements may be identified by a common reference character or numeral, with suffixes being used to refer to specific occurrences of the element. Examples given in this application are prophetic examples.

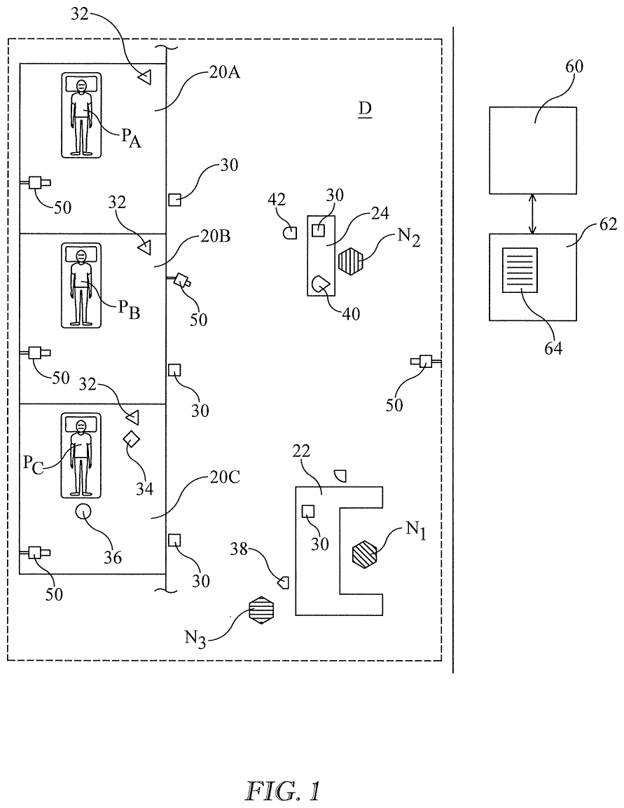

FIG. 1 is a schematic plan view of a hospital wing. The illustrated wing is an example to disclose the features of the methods and devices that are the subject of this application. In general, the principles of the invention disclosed herein can be applied to larger or smaller areas. The area to which the invention is applied is referred to as a domain D. The domain of FIG. 1 is surrounded by a dashed line border.

The illustrated domain or hospital wing includes three patient rooms 20A, 20B, 20C each having a bed. The wing includes a nurses' station 22 and an auxiliary work station 24. The wing includes various items of equipment indicated schematically by different symbols. Example equipment includes hand sanitizer dispensers 30, a wall set 32 of diagnostic instruments (e.g. blood pressure cuff, thermometer, blood oxygen saturation sensor) in each patient room, IV poles 34, oxygen tanks 36, respiratory assistance devices 38, an ophthalmoscope 40, and a rolling cart 42. Other equipment in the domain includes cameras 50. Some of the equipment may not be movable from place to place, for example some of the hand sanitizer dispensers may be mounted on a wall. Other equipment may be movable, for example by virtue of being mounted on casters, for example cart 42. Other equipment may be movable by virtue of being small and light enough to carry by hand, for example ophthalmoscope 40.

FIG. 1 also shows a processor 60, a memory 62, and a set of machine readable instructions 64 stored in the memory.

FIG. 1 also shows a patient P occupying each bed, and three staff members such as nurses N₁, N₂, N₃ depicted schematically by hexagon symbols. The patients and nurses are members of a population who are at risk of acquiring a communicable condition from another person in the wing. The population of interest is not limited to the individuals shown in the illustration, but also includes other persons who later enter the domain or who may have been in the domain but are no longer present. The population may include other patients, other medical staff, maintenance staff, and housekeeping staff to name just a few. If desired, the method described herein may selectively exclude one or more individuals from the population even though those individuals may be in the domain, may have been in the domain in the past but are no longer present, or may enter the domain in the future.

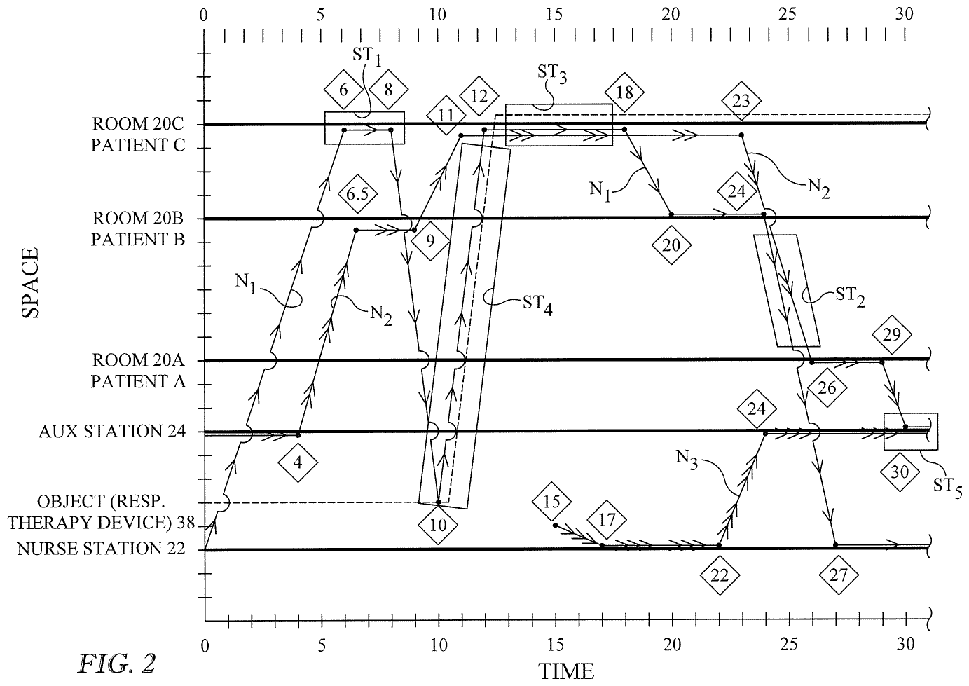

FIG. 2 is a space/time diagram showing a sample of the locations and movements of objects and nurses during a period of time such as part of a typical work shift. The solid lines without arrowheads represent objects which, according to this example, are nonmovable. These objects include nurses' station 22, auxiliary station 24 and patient rooms 20A, 20B, 20C. The dashed line represents an object which is movable, for example respiratory therapy device 38. The lines with single, double and triple arrowheads represent nurses N₁, N₂ and N₃. Each line is a space/time trajectory, or simply trajectory. The trajectory of a member of the population or a movable object can be thought of as the space/time coordinates of that member or object. The trajectory for each nonmovable object may be thought of as the space/time coordinates of that object. Because the object is immovable its spatial coordinate is constant over time and is therefore represented by a horizontal line in the diagram. For ease of reference, the diagram includes diamond symbols displaying the time coordinates of events described below.

The time scale on the horizontal axis of the diagram is for convenience of referring to events, but is not intended to show realistic units of time. Moreover, because the two dimensional area of the hospital wing is represented as a single dimension on the diagram, the coordinates where a nurse's trajectory and an object's trajectory cross each other do not necessarily represent an interaction between the nurse and the object. Such lack of interaction is indicated by small arch-like bridges or humps in the nurse's trajectory. For example the arch at t=4.5 of nurse N₂'s trajectory shows that the nurse did not stop at room 20A when traveling from the auxiliary station to patient room 20B.

In the example of FIG. 2, nurse N₁ proceeds from the nurses' station 24 to room 20C. Two time units after arriving at room 20C the nurse leaves the room and, at t₁₀, arrives at the location of respiratory therapy device 38. The nurse retrieves the device and returns to room 20C, arriving at time t₁₂. Nurse N₁ departs room 20A at t₁₈, leaving device 38 in the room. At t₂₀ nurse N₁ arrives at room 20B. The nurse remains at room 20B until t₂₄, then departs and arrives back at nurses' station 22 at t₂₇.

Nurse N₂ is at auxiliary station 24 from t₀ to t₄, at which time nurse N₂ departs for room 20B. Nurse N₂ arrives at room 20B at t_{6.5} and remains there until t₉. Nurse N₂ then travels to room 20C, arriving there at t₁₁. Nurse N₂ departs room 20B at t₂₃ and arrives at room 20A at t₂₆. At t₂₉ the nurse leaves room 20A and arrives at auxiliary station 24 at t₃₀. During part of that time (from t₂₄ to t₂₆) nurse N₁ and nurse N₂ are in each other's presence.

Nurse N₃ enters domain D at t₁₅ in the vicinity of nurses' station 22. Nurse N₃ proceeds toward the nurses' station. The nurse arrives at the nurses' station at t₁₇ and remains there until t₂₂. From t₂₂ to t₂₄ nurse N₃ travels from the nurse's station 22 to auxiliary station 24 and remains there for at least the duration of time depicted in the chart.

The trajectories of people and objects, such as those described above, may expose the members of the population to communicable conditions. As used herein a communicable condition is a condition, for example a disease or other medical disorder (or a cause of the condition such as bacteria or virus) that can be transferred, transmitted or otherwise propagated from one member of the population to another.

The transference of the communicable condition can be direct. One example of direct transference is actual physical contact between a first member of the population affected by the condition and a second member. Another example of direct transference is by droplets or aerosol, for example due to the affected member coughing or sneezing and the recipient member being exposed to the resultant aerosol or droplets. Another example of direct transference is by way of the recipient member being in direct contact with a bodily fluid (e.g. blood, mucus) of an affected member. As is evident from the sneeze and bodily fluid examples, direct transference need not involve physical bodily contact between the two members. However in all cases, direct transference involves only a first (source) person and a second (recipient) person. In other words direct transference does not require a third member, or an object, to serve as a mediator.

One example of a possible direct transference is shown at space/time block ST₁ of FIG. 2. Nurse N₁ is in close proximity to patient P_{c} and therefore a communicable condition can be transmitted between them, for example from the patient to the nurse. Another possible direct transference is shown at ST₂ where nurses N₁ and N₂ are walking together and a communicable condition can be transmitted between them, for example from nurse N₂ to nurse N₁.

The transference of the communicable condition may also be indirect transference by way of a mediator. A mediator may be a linking object or may be a linking member of the population. Indirect transference involves direct transfer of the condition (or a cause of the condition such as bacteria or virus) from a source member to a mediator and subsequent direct transference from the mediator to the recipient member. One example of indirect transference is if a first member who has the condition sneezes on an object and, at a later time, a different member touches the same object, thereby becoming the recipient for the communicable condition. A similar example of indirect transference is if the first member (the member with the condition) touches a different member (e.g. shaking hands) thereby contaminating that member and, at a later time, a second member touches that different member, thereby becoming the recipient or recipient for the communicable condition.

An example of indirect transference is shown at space/time block ST₃ in conjunction with space/time block ST₄. At ST₄ nurse N₁ may transfer the cause of a communicable condition (e.g. a virus) to the respiratory care device 38 retrieved at time t₁₀. At ST₃ nurse N₂ may handle the same object, thereby becoming the recipient of the virus and therefore the recipient for the communicable condition caused by the virus. The indirect transference is a chain of transference from N₁ to the object to N₂, with the object serving as a mediator. Another example of indirect transference is shown at ST₅ in conjunction with ST₂. At ST₂ nurse N₁ may transfer a communicable condition to nurse N₂. At space/time block ST₅ nurse N₂ may transfer that condition to nurse N₃. Thus, the chain of transference is from N₁ to N₂ to N₃ with N₂ serving as a mediator.

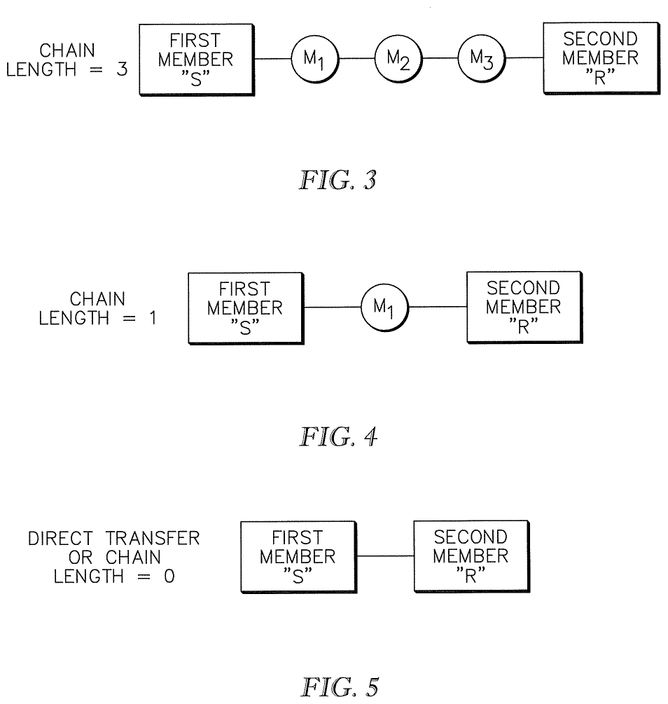

A transference chain such as those described above has a chain length. The chain length may be defined as the quantity of mediators plus two (one for the first member; one for the second member) so that the chain length accounts for the first and second members. Another possible definition of chain length is the quantity of mediators (the first and recipient members are excluded from the length). The explanations in this application adopt the latter definition. Under that definition, direct transference may be thought of as a limit case of indirect transference in which the chain length is zero. Accordingly, the chain of FIG. 3 has a length of three. The chain of FIG. 4 has a length of one. If the example of FIG. 5 is considered to be a chain, its chain length is zero.

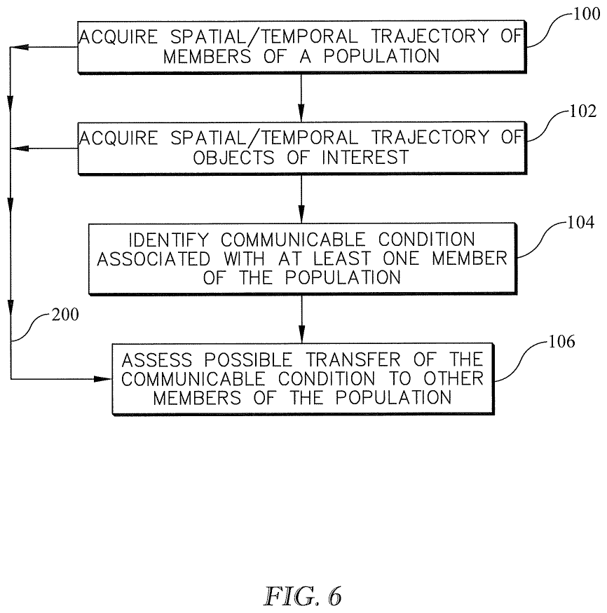

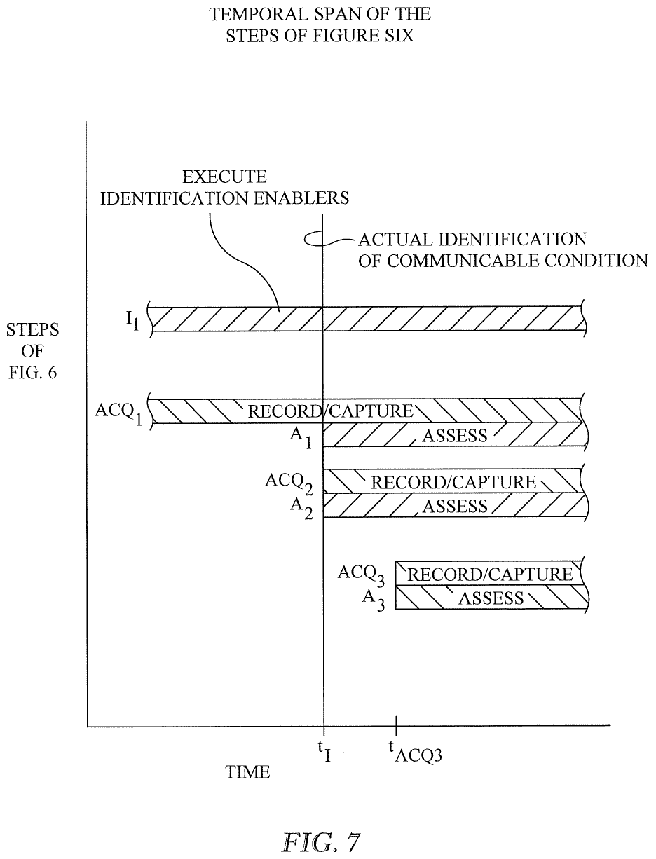

FIG. 6 illustrates a method of responding to the presence of a communicable condition. At block 100 the method acquires the spatial/temporal trajectory of each member of a population of interest in domain D. The acquisition of the trajectories may be carried out by any suitable method, for example by analyzing images captured by cameras 50, by tracking the locations, over time, of RFID tags which are worn by members of the population and affixed to objects, or by any other locating and tracking means. Trajectories of the population members may be recorded for future consultation, but need not be.

At block 102 the method acquires the spatial/temporal trajectory of each object of interest in domain D. The acquisition of the trajectories may be carried out by any suitable method, for example by analyzing images captured by cameras 50, by tracking the locations, over time, of RFID tags which are worn by members of the population and affixed to objects, or by any other locating and tracking means. Trajectories of the objects may be recorded for future consultation, but need not be. Blocks 100 and 102 are arranged in series, however the acquisitions may be carried out contemporaneously or in the opposite order.

At block 104 the method identifies a communicable condition associated with at least one first member of the population. In particular the method identifies a source member. The source member is a member of the population who is considered to have introduced the condition into the domain, as distinct from having acquired it while in the domain.

Determining whether a member of the population is the source of a communicable condition may be done in any suitable way. For example conventional diagnosis techniques such as examination by a physician, imaging, and laboratory tests may be used to identify a member as the source of a condition. In another example members in domain D may be outfitted with monitors that collect physiological information which is analyzed by a software program to determine if a member is affected by a communicable condition. In yet another example, cameras 50 may be used to collect images of members in the domain. A software program analyzes the images for evidence that the member is affected by a particular condition. Such evidence may include factors such as pale skin, excessive perspiration, and sluggish behavior. The foregoing methods and other methods of determining if a member has a communicable condition can be used independently or in various combinations.

In connection with the step at block 104, “identify” and related words may refer to the actions, such as those enumerated above, carried out in order to determine if a member of the population is a “source” of a particular communicable condition. “Identify” may also refer to the act of reaching a conclusion, based on the examination, testing, analyses, etc., that a member is the source of a communicable condition. Therefore, “identify” and related words may refer to the actual conclusion that the member is the source of a communicable condition, to the actions carried out earlier in time which enable the conclusion to be reached, or to both. To the extent that it is necessary to distinguish the moment and/or act of reaching a conclusion from the enabling activities or enablers, that distinction will be clear from context.

At block 106 the method assesses the possibility that the communicable condition identified at block 104 may have been transmitted to other members of the population. As indicated by arrow 200, the assessment depends at least partly on the trajectories acquired at blocks 100 and 102. Referring additionally to FIG. 2, assume that block 104 determines that patient P_{c} has a communicable condition C₁, but does not reach that conclusion until time t₃₀. Providing that the incubation period of the condition is at least as long as the twelve unit time interval from t₁₈ to t₃₀, the method concludes that nurse N₁'s contact with patient P_{c} at time t₁₈ (and possibly earlier depending on the incubation period) has caused nurse N₁ to be at risk of having acquired condition C₁ from patient P_{c}. By using the trajectories of FIG. 2, the method may reach one or more of the conclusions listed below.
A) Patient P_{c} may have transferred condition C₁ to nurse N₁ and/or to nurse N₂ at or before time t₁₈ due to the nurses' presence in room 20C and the nurses' presumed or confirmed interaction with patient P_{c}.
B) If either nurse N₁ or nurse N₂ acquired the condition from patient P_{c}, the affected nurse (N₁ or N₂) may have transferred the condition to the other nurse (N₂ or N₁) during their interaction from t₂₄ to t₂₆
C) If nurse N₂ acquired the condition from patient P_{c} or from nurse N₁, nurse N₂ may have transferred that condition to nurse N₃ due to their interaction at auxiliary station 24 at t₃₀ to t₃₁.

If the incubation period is longer than the twelve unit time difference from t₁₈ to t₃₀, for example the twenty four time unit difference from t₆ to t₃₀, and still assuming that P_{c} is not identified as a source of the condition until t₃₀, other conclusions that may be reached include those listed below.
D) Patient P_{c} may have transferred the communicable condition to nurse N₁ when the nurse visited room 20B from t₆ to t₈.
E) If nurse N₁ acquired the communicable condition from patient P_{c}, in the interval t₆ to t₈, nurse N₁ may have subsequently transferred the condition to device 38 at some time after t₁₀.

The conclusions A through E listed above are not an exhaustive list of all possible conclusions that may be reached. That is, they are not an exhaustive set of assessments concerning possible transfer of the communicable condition to one or more other members of the population. Instead they are examples illustrating that the assessment is based on the spatial/temporal trajectories of the members of the population and the spatial/temporal trajectories of the objects of interest. Moreover, the examples demonstrate that the assessment of possible transfer of the communicable condition is based, at least in part, on an interaction or interactions along the trajectory. The interaction or interactions are between a first member of the population and a second member of the population or between a member of the population and an object.

The above examples also demonstrate that the assessment can include a component which is prospective in relation to the time t₃₀ at which the communicable condition is first recognized (e.g. nurse N₃ acquired the condition from nurse N₂ sometime after t₃₀ while the nurses were together at auxiliary station 24). The prospective component is valuable because it will ordinarily not be possible to take immediate action to prevent further propagation of the condition. The prospective component provides knowledge of any propagation that occurs before such action gets underway.

Alternatively or additionally the examples demonstrate that the assessment can include a component which is retrospective in relation to the time t₃₀ at which the communicable condition is first recognized (examples of such a retrospective component include examples A, B, D and E, above). In general the assessment may have both prospective and retrospective components.

The examples also demonstrate that the assessment may be based on the quantity and duration of interactions, including:
a) the quantity and/or duration of one or more direct member to member interactions,
b) the quantity and/or duration of one or more indirect member to member interactions,
c) the quantity and/or duration of one or more direct member to object interactions; and
d) the quantity and/or duration of one or more indirect member to object interactions.
   For example, a larger number of interactions between a person and an object to which a disease causing agent (e.g. a virus) has been transferred) is expected to be more likely to result in that person's acquisition of the condition than would be the case for a smaller number of interactions. Similarly, longer duration interactions are expected to be more effective at transferring the condition than shorter duration interactions.

Another consideration that assessment step 106 may account for is the length of the chain involved in an indirect transference of a communicable condition. A longer chain may be less likely than a short chain to transfer a communicable condition. For example in the three mediator chain of FIG. 3, mediator M₁ may be at relatively high risk of acquiring a communicable condition from member S. Mediators M₂, M₃ and member R may be at progressively diminishing risk, with member R being at the least risk.

Another consideration that assessment step 106 may account for is a sanitizing event. One example of a sanitizing event is a cleansing procedure carried out by a member of the population on himself or herself, for example washing his hands or applying hand sanitizer from one of dispensers 30. For example if nurse N₁ had used sanitizer when departing room 20C at t₁₈ the likelihood of transmitting a communicable condition to nurse N₂ at time t₂₄ to t₂₆ might have been reduced. Similarly, sanitizing respiratory therapy device 38 after using it in room 20C may have reduced the likelihood of transferring a communicable condition to nurse N₂ during that nurse's presence in room 20C.

Block 104 of FIG. 3 is arranged in series with blocks 100 and 102, however in practice the temporal relationship between identification step 104 and acquisition steps 100, 102 need not be as illustrated. FIG. 7 shows an example in which the actual identification of the communicable condition and its association with a first member of the population occurs at time t_{I}. As illustrated, the method has been executing the actions to enable the identification of the condition since at least t_{I}, and possibly since before t_{I} (bar I₁). Execution of the enabling action is shown as continuing after t_{I} in order to continue surveying the population to identify a possible second occurrence of the same condition (associated with a second member) or to identify a possible occurrence of a different communicable condition associated with either the first member or the second member.

Bar ACQ₁ shows an option of acquiring spatial/temporal trajectories prior to t_{I}. One reason to acquire trajectories before t_{I} is that this “historical” information, if recorded for later use, can facilitate a temporally backward looking or retrospective assessment at block 106. The ability to carry out a retrospective assessment could be of value because the source member may have been capable of transferring the condition to another member or object before there had been any recognition that the source member was affected by the condition. That is, the source member may have been an asymptomatic carrier of the condition prior to t_{I}. Of course, the assessment itself cannot commence until the condition is identified and associated with a member of the population at t_{I} (bar A₁).

Bar ACQ₂ shows an option in which trajectory acquisition does not commence until t_{I}, the time of actual identification of the communicable condition. As with the immediately preceding example, the assessment itself (bar A₂) cannot commence until at least t_{I}. Unlike the immediately preceding example, the assessment A₂ cannot be retrospective with respect to t_{I}, even if the trajectories acquired during ACQ₂ are recorded.

Bar ACQ₃ shows an option in which trajectory acquisition does not commence until t_{ACQ3}, which is after time t_{I}, the time of actual identification of the communicable condition. The assessment itself (bar A₃) cannot commence until at least the onset of acquisition, t_{ACQ3}. The assessment will suffer from not having the benefit of trajectory information in the time frame from t_{I} to t_{ACQ3}. Such “lost” information could compromise the accuracy of the assessment because interactions that transfer the communicable condition to other members or objects could have taken place in the time frame t_{I} to t_{ACQ3}.

Assessments A₁ and A₂ may be delayed until a time later than t_{I}. Assessments A₂ and A₃ may be delayed until a time later than the time at which trajectory acquisitions ACQ₂, ACQ₃ begin. To the extent that the acquired trajectories are stored and available for use, the method can still use that stored information in the assessments.

In all cases, if the trajectories are not stored, the method will use the acquired trajectories to carry out a real time assessment of possible transference of the communicable condition.

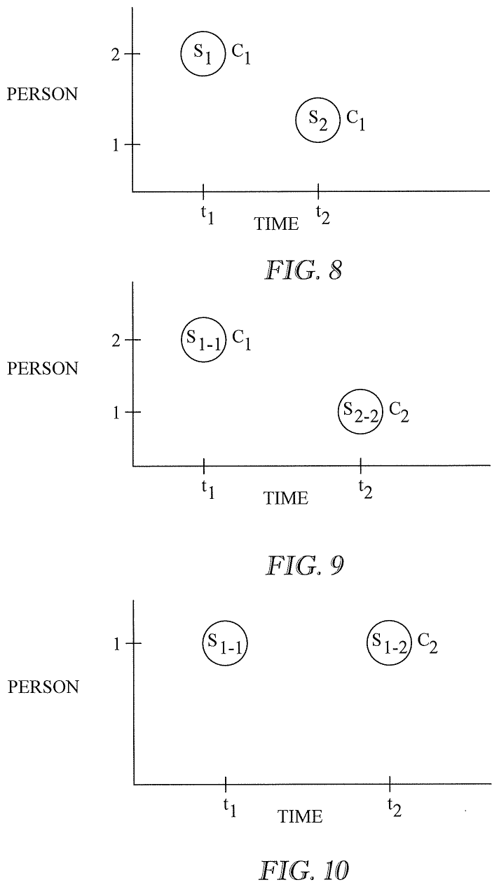

As described thus far the method accounts for a single source member of the population and a single communicable condition which affects that member. However the method may also account for more general occurrences. In the example of FIG. 8, the method associates a communicable condition with a first member of the population and with a second member of the population. At time t₁ the method identifies a member S₁ as a first source member with respect to communicable condition C₁. At time t₂ the method identifies a member S₂ as a second source member with respect to that same communicable condition C₁. In general t₁ is not equal to t₂, but may be. Both S₁ and S₂ are source members. S₁ may also be considered to be a population member other than a source member with respect to S₂. S₂ may be considered to be a population member other than a source member with respect to S₁. Such dual identity may or may not be meaningful in assessing transfer of the communicable condition.

FIG. 9. shows another example of an occurrence more general than a single source member and a single communicable condition. In the example of FIG. 9, the method associates a first communicable condition with a first member and associates a second communicable condition with a second member. At time t₁ the method identifies a member S₁₋₁ as a first source member with respect to communicable condition C₁. At time t₂ the method identifies a member S₂₋₂ as a second source member with respect to a different communicable condition C₂. In general t₁ is not equal to t₂, but may be. S₁₋₁ may also be considered to be a population member other than a source member with respect to condition C₂. S₂₋₂ may be considered to be a population member other than a source member with respect to condition C₁. Such dual identity may be meaningful in that member S₁₋₁, in his capacity as a source for communicable condition C₁, could transfer that condition to member S₂₋₂. Similarly, member S₂₋₂, in his capacity as a source for communicable condition C₂, could transfer that condition to member S₁₋₁.

Another example of an occurrence more general than a single source member and a single communicable condition is shown in FIG. 10. In the example of FIG. 10 the method associates a first condition and a second condition with the same member of the population. At time t₁ the method identifies a member S₁ as a source member with respect to communicable condition C₁. At time t₂ the method identifies that same member S₁ as a source member with respect to communicable condition C₂. In general t₁ is not equal to t₂, but may be.

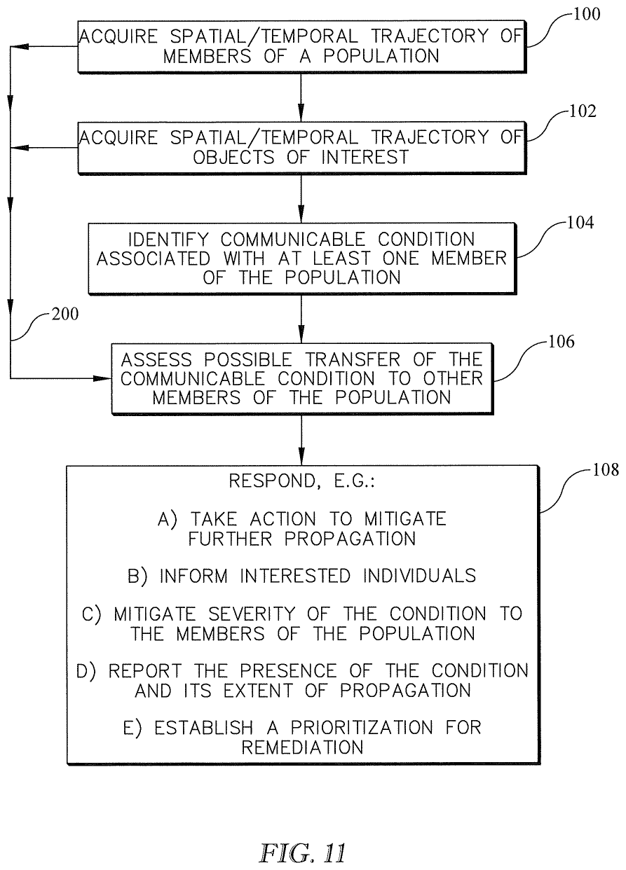

FIG. 11 is a block diagram similar to FIG. 6, but with a post-assessment step 108 of responding to the assessment of a possibility that a communicable condition has been transferred to other members of the population.

One example of a response is taking an action to mitigate further propagation of the condition. Such an action might take the form of removing the source member from the domain.

Another example of a response is informing interested individuals of at least the presence of the condition. One example of an interested individual is a member of the population who the assessment shows to have been placed at risk of acquiring the communicable condition. Another example is a facility manager having responsibility for controlling or containing outbreaks of communicable conditions.

Another example of a response is taking an action to mitigate the severity of the condition to the members of the population who the assessment step has identified as having acquired the condition or being at risk of acquiring the condition. One example of a mitigation response is vaccinating exposed members of the population, provided that the communicable condition is one for which a vaccine is available and that post-exposure administration of the vaccine is effective at preventing the member from becoming symptomatic or reducing the severity of the condition if it does develop.

Another example of a response is reporting the presence of the condition and its extent of propagation, for example by a general announcement not restricted to members of the population.

Another example of a response is establishing a prioritization for remediation, in other words establishing a schedule for removing the source member from the domain, notifying interested individuals, vaccinating exposed members, ensuring that exposed members are cleansed of the condition, and cleaning affected objects.

Referring back to FIG. 1, an apparatus for detecting and responding to the presence of a communicable condition includes processor 60 and machine readable instructions 64 which are executable by the processor. The instructions are held in a memory 62. The instructions, when executed by the processor, carry out at least the operations of blocks 100, 102, 104, 106 of FIG. 6. This may alternatively be though of as the processor, when acting according to instructions, carrying out the operations of blocks 100, 102, 104, and 106. The instructions, when executed by the processor, may also carry out selected operations enumerated in block 108 of FIG. 11. For example operations B, D and E of FIG. 11 are operations that the processor and instructions may be able to carry out automatically.

Although this disclosure refers to specific embodiments, it will be understood by those skilled in the art that various changes in form and detail may be made without departing from the subject matter set forth in the accompanying claims.

1. A method of responding to the presence of a communicable condition comprising:
acquiring a spatial/temporal trajectory of each member of a population of interest;
acquiring a spatial/temporal trajectory of zero or more objects of interest;
identifying a communicable condition associated with at least a first member of the population; and
subsequent to the identifying step, assessing possible transfer of the communicable condition to one or more other members of the population, the assessment being based, at least in part, on the acquired spatial/temporal trajectories.

2. The method of claim 1 wherein at least a portion of the acquired trajectory is stored and is recallable for later use.

3. The method of claim 1 wherein the assessment is based, at least in part, on an interaction or interactions along the trajectory, each interaction being between:
A) a first member of the population and a second member of the population, or
B) a member of the population and the object.

4. The method of claim 1 wherein the assessment includes a prospective component and a retrospective component.

5. The method of claim 1 wherein the identifying step associates the communicable condition with a first member of the population and with a second member of the population.

6. The method of claim 1 wherein the identifying step associates a first communicable condition with a first member and associates a second communicable condition with a second member.

7. The method of claim 1 wherein the identifying step associates a first condition and a second condition with a first member of the population.

8. The method of claim 1 wherein the assessment is based on one or more of:
A) quantity and/or duration of one or more direct member to member interactions;
B) quantity and/or duration of one or more indirect member to member interactions;
C) quantity and/or duration of one or more direct member to object interactions; and
D) quantity and/or duration of one or more indirect member to object interactions.

9. The method of claim 8 wherein each indirect interaction is mediated by at least one mediator, and wherein the at least one mediator is:
A) one or more linking members; and
B) one or more linking objects.

10. The method of claim 9 wherein the at least one mediator links a first member to a second member, the linking member, first member and second member define a chain having a length, and the assessment takes into account the chain length.

11. The method of claim 1 wherein the assessment accounts for a sanitizing event.

12. The method of claim 11 wherein the sanitizing event comprises:
A) a cleansing procedure carried out by a member of the population on himself or herself, and/or
B) sanitizing of an object in the domain.

13. The method of claim 1 comprising at least one of:
A) taking an action to mitigate further propagation of the condition;
B) informing interested individuals of at least the presence of the condition;
C) taking an action to mitigate the severity of the condition to the members of the population who the assessment step has identified as having acquired the condition or being at risk of acquiring the condition;
D) reporting the presence of the condition and its extent of propagation;
E) establishing a prioritization for remediation.

14. An apparatus for responding to the presence of a communicable condition comprising:
a processor; and
machine readable instructions which, when executed by the processor result in:
acquisition of a spatial/temporal trajectory of each member of a population of interest;
acquisition of a spatial/temporal trajectory of zero or more objects of interest;
identification of a communicable condition associated with at least a first member of the population; and
subsequent to the identifying step, assessment of possible transfer of the communicable condition to one or more other members of the population, the assessment being based, at least in part, on the acquired spatial/temporal trajectories.

15. The apparatus of claim 14 wherein the machine readable instructions, when executed by the processor, cause at least a portion of the acquired trajectory to be stored and to be recallable for later use.

16. The apparatus of claim 14 wherein the machine readable instructions, when executed by the processor, cause the assessment to be based, at least in part, on an interaction or interactions along the trajectory, each interaction being between:
A) a first member of the population and a second member of the population, or
B) a member of the population and the object.

17. The apparatus of claim 14 wherein the machine readable instructions, when executed by the processor, include a prospective component and a retrospective component in the assessment.

18. The apparatus of claim 14 wherein the machine readable instructions, when executed by the processor, cause the assessment to be based on one or more of:
A) quantity and/or duration of one or more direct member to member interactions;
B) quantity and/or duration of one or more indirect member to member interactions;
C) quantity and/or duration of one or more direct member to object interactions; and
D) quantity and/or duration of one or more indirect member to object interactions.

19. The apparatus of claim 14 wherein the machine readable instructions, when executed by the processor, account for a sanitizing event when making the assessment.

20. The apparatus of claim 14 wherein the machine readable instructions, when executed by the processor, result in at least one of:
A) taking an action to mitigate further propagation of the condition;
B) informing interested individuals of at least the presence of the condition;
C) taking an action to mitigate the severity of the condition to the members of the population who the assessment step has identified as having acquired the condition or being at risk of acquiring the condition;
D) reporting the presence of the condition and its extent of propagation; and
E) establishing a prioritization for remediation.
